(19) United States
(12) Patent Application Publication
(10) Pub. No.: US 2023/0348839 A1
(43) Pub. Date: Nov. 2, 2023

(54) TREATMENT OF A MASS OF MATERIAL BY PRESSING

(71) Applicant: Taprogge GmbH, Wetter (DE)

(72) Inventors: Simon Höckenkamp, Lüdinghausen (DE); Dietmar Altegoer, Witten (DE); Detlef Taprogge, Hagen (DE); Markus Barthelmes, Witten (DE)

(21) Appl. No.: 18/131,026

(22) Filed: Apr. 5, 2023

(30) Foreign Application Priority Data
Apr. 5, 2022 (DE) 10 2022 108 172.9

(51) Int. Cl. C12M 1/33; B30B 9/06; C12M 1/34; B30B 15/30; C12M 1/107; C12M 1/00

(52) U.S. Cl. CPC C12M 45/02; B30B 9/06; B30B 15/30; C12M 21/04; C12M 41/12; C12M 45/03

(57) ABSTRACT

--A device and method for dewatering a mass of material and a method and system for recycling a biogenic material. A vertical press has a press space formed between a tool provided with openings and a press plunger movable relative thereto. Conveying means convey the mass of material from a feed side toward an opposite discharge side. The press space is laterally confined by opposite side walls but is not confined in the direction of the feed side and the discharge side. A biogenic material is converted microbially and/or enzymatically into gas and fermentation residues in a fermentation plant and the latter are dewatered by means of a vertical press to such an extent that thermal utilization is made possible.--

### CROSS-REFERENCE TO RELATED APPLICATIONS

This application claims the benefit of Germany Patent Application No. 10 2022 108 172.9, filed Apr. 5, 2022, which is incorporated herein by reference in its entirety.

### FIELD OF THE INVENTION

The invention relates to a device and a method for dewatering a mass of material and to a method and a system for recycling a biogenic material. In particular, the invention relates to devices and methods in which a press is used for dewatering.

### BACKGROUND OF THE INVENTION

US 4,630,535 describes a device and method for pressing moisture out of wood chips and other biomass material. A press includes a closed chamber and a ram. A charge of biomass is periodically introduced for dehydration. The ram is guided against the biomass with sufficient force to compress it, causing liquid to flow through grooved walls of the chamber. Thus, moisture is squeezed out of the biomass and flows through the grooves into a collection container.

EP 2 176 061 B1 describes the compression of biomass and other carbonaceous materials for high energy fuels. A compactor contains carbonaceous material and provides blocks formed therefrom that can be used for power generation and as feedstock for ethanol production and gasification. A compression chamber for the carbonaceous material has one or more rams used to apply pressure to the material, a plate against which the material is compressed, and one or more drainage ports or a membrane that allows liquid to pass through.

It can be considered as an object to propose a device and a method for dewatering a mass of material as well as a method and a system for recycling a biogenic material, in which a particularly efficient dewatering is made possible.

### SUMMARY OF THE INVENTION

The object is solved according to a first aspect by a device and a method for dewatering a mass of material, and according to a second aspect by a method and a system for recycling a biogenic material. The aspects of the invention are each advantageous individually, but develop particular advantages in combination. Dependent claims relate to advantageous embodiments of the invention.

In the first aspect of the invention, the dewatering of a mass of material, in particular a biomass, is performed by means of a vertical press.

The vertical press has a tool provided with openings and a press plunger which is movable relative to the tool. A press space is formed between the tool and the press plunger. A press space can be understood as a preferably cuboidal space which is defined in lateral extension by the overlapping of the tool and the press plunger.

In the vertical press, the press plunger preferably moves along a linear path and, more preferably, at least essentially in a vertical direction, which means that slight inclinations (e.g. up to 30°, preferably no more than 15°) are also considered to be included. Particularly preferably, the tool is located at the bottom in relation to the press space and the press plunger moves from above in the direction of the tool during pressing. During pressing of an aqueous mass of material located in the press space, liquid contained therein and pressed out during the pressing process can drain off through the openings of the tool.

The device according to the invention has conveying means for conveying the mass of material from a feed side into the press space and for conveying the pressed mass of material out of the press space in the direction of a discharge side opposite the feed side. Preferably, the mass of material is conveyed in an at least substantially horizontal direction, which is to be understood as to also include slight inclinations (for example up to 30°, preferably not more than 15°).

The press space is laterally confined by opposite side walls which extend between the feed side and the discharge side and may, for example, extend beyond the press space on one or both sides. In contrast, there is no lateral boundary in the direction of the feed side and the discharge side, so that the press space is open towards the feed side and discharge side. Preferably, the press space is open over its entire width and/or over its entire height towards the feed side and discharge side in each case.

The method according to the invention and the device according to the first aspect are suitable for very efficient dewatering of a mass of material, and thus in particular for treating a relatively large stock quantity per unit of time. Due to the press space being open to the feed side and discharge side in each case, a fast feed and discharge is possible, which makes a fast working cycle possible. Thereby, the vertical press used according to the invention can apply considerable pressing forces, enabling fast and efficient dewatering of the mass of material. In particular, in the case of a mass of material with an initially high liquid quantity, for example a dry substance (DS) value of less than 50%, preferably less than 40%, further preferably even 30% or less, substantial dewatering to DS values of more than 50%, preferably more than 55%, and particularly preferably more than 60% is possible in the shortest possible time by means of a press stroke. The press stroke can be performed continuously at the same press pressure or speed, but it is also possible for a feed and/or pressure profile to be specified and controlled, for example, by a press control system. Such a profile can have sections with faster and slower feed or higher or lower pressure and, if necessary, also standstill intervals to allow sufficient time for the liquid to drain off.

According to an advantageous further development of the invention, the conveyor means may comprise at least one conveyor belt, in particular preferably a liquid-permeable conveyor belt. For example, the conveyor belt can be designed as a liquid-permeable membrane or be provided with openings.

The conveyor belt can, for example, extend from the feed side to the discharge side and rest in the press space on the tool provided with openings. While it is possible for a conveyor belt to only partially cover the length and/or width of the press space, the conveyor belt can preferably cover the entire press space in length and/or width throughout, thus providing for full-area, continuous transport. To enable continuous operation, the conveyor belt is preferably closed by a return run outside the press space.

The side walls of the press space and/or the surface of the press ram facing the press space can, for example, have a closed, e.g. plane surface. Preferably, they can be grooved and/or provided with openings to support the discharge of pressed-out liquid. In the case of larger quantities of liquid, such openings can contribute to effective dehumidification despite the possibility of partial rewetting. A filter element such as a filter layer or a sieve can be arranged in or in front of the openings made in the respective surfaces, e.g. holes, in order to allow liquid to pass through without solid components being able to settle in the openings and block them. To prevent clogging of the openings, another liquid-permeable conveyor belt may also be provided. It is also possible for discharge and/or suction devices to be arranged at the openings in order to discharge liquid emitting there, for example to discharge it downward by gravity or to actively suck it off in order to prevent backflow. In the case of a targeted discharge of accumulating liquids, such as active suction, rewetting can be reduced.

According to a further development of the invention, the tool has a structure comprising a perforated plate with through-openings, a base plate and a support frame arranged between the perforated plate and the base plate, so that a flow space is formed for discharging liquid through the openings and through the interior of the support frame. The support framework may, for example, include a plurality of supports distributed over the area of the perforated plate, which may extend individually between the perforated plate and the base plate or may be transversely connected. The supports can be plate-shaped, for example.

Preferably, collecting means and at least one conduit can be provided for collecting and for directing liquid pressed through the openings. The collecting means may be provided, for example, by the space formed underneath a perforated plate if this space is designed and preferably closed in such a way as to allow selective directing of emitting liquid into an outlet connected to a conduit. The conduit is preferably a down-feed line so that a slope can be used to discharge the liquid. It may be preferred that the tool protrudes laterally in one or more directions, or that other protruding collecting means are provided to also collect portions of liquid that emit outside the press space. In the case of liquids occurring outside the die plate, protruding collecting means can also absorb and drain off these components.

A scraper can be provided on the feed side to limit a filling height of the mass of material when it is fed in. The scraper can preferably be arranged at a fixed distance above the tool and, if necessary, a conveyor belt running on it, and thus specify the maximum filling height. Portions of the mass of material that exceed the predetermined maximum filling height are then stripped off and retained. In this way, a stroke path of the die can be favorably dimensioned and well utilized.

According to a preferred embodiment, an arrangement for pre-compacting the mass of material may be provided on the feed side. This can be a press arrangement with a precompression plunger which preferably moves on a linear path, particularly preferably parallel to the path of the press plunger, relative to a precompression tool, whereby preferably the precompression tool can be identical to the tool of the vertical press. Further preferably, the pre-compacting also takes place on the conveyor belt. Pre-compacting is preferably carried out at a lower pressing pressure than the subsequent pressing, and preferably results in a reduction in the filling height of the mass of material, - as the case may be, in addition to an initial partial dewatering. In this way, the stroke of the press plunger of the vertical press can be limited and used efficiently. The arrangement for pre-compacting can be coupled with the vertical press, so that the pre-compacting of a part of the mass of material takes place simultaneously with the pressing of another, in particular already pre-compacted part of the mass of material. The coupling can be achieved, for example, by mechanical coupling to the press plunger. It is also possible that the pre-compacting arrangement can be controlled and/or driven separately from the press plunger of the vertical press.

According to a second aspect of the invention, the method and the system comprehensively utilize a biogenic material. Biogenic materials are understood to be materials that contain biomass or are produced from biomass by upstream processing steps. Typical biogenic substances may be of animal and/or plant origin, for example, and may include, for example, parts of plants, organic waste and organic residues, for example, from agriculture, gastronomy, the food industry and domestic, commercial and industrial use.

Gas is first extracted from the biogenic material in a fermentation plant, in particular gas containing methane. This produces fermentation residues. In this context, the term fermentation plant is understood to mean any known form of plant in which organic substances are converted by means of biological processes, e.g. microbially and/or enzymatically. This includes all types of fermentation including fermentation or comparable biological processes, with or without the inclusion of oxygen. In particular, known biogas plants are included.

A (bio)gas, in particular a methane-containing gas, is produced in the fermentation plant. The resulting fermentation residues are initially in solid/liquid mixed form and -if necessary after prior solid/liquid separation - are dewatered in accordance with the invention by means of a vertical press to such an extent that thermal utilization is possible. In addition to combustion, thermal utilization also includes processes such as pyrolysis. Thermal utilization is given if the thermal utilization process (for example combustion in a combustion chamber) releases more energy than is required to achieve combustibility.

Such a method and system according to the second aspect of the invention enables highly efficient operation by using a vertical press. This allows high pressing forces to be applied, with which dewatering, in particular of fermentation residues with typically high moisture content, is possible. The aim is not necessarily to achieve very high degrees of dryness, e.g. more than 90%, but rather to close the gap between typically high to very high moisture content of the fermentation residues to dry substance values that allow thermal utilization. For example, the fermentation residues can be dewatered by means of the vertical press to a dry substance value of 50 to 90%, preferably 55 to 80%, particularly preferably 60 to 75%. In the value ranges mentioned, the respective lower limit is decisive for thermal utilization being possible or reasonably efficient, while the respective upper limit indicates a degree of dryness which can still be achieved efficiently by means of a vertical press.

According to a particularly preferred further development of the second aspect of the invention, press water pressed out by the vertical press is fed back to the fermentation plant and/or to a treatment in which, for example, ingredients are extracted. The utilization of press water by treatment and, in particular, the return of press water to the fermentation plant enables a particularly efficient utilization of the biogenic material. An otherwise necessary disposal of the press water via the public sewer with subsequent biological pollution can then be omitted, or at least significantly reduced.

Another particularly preferred further development provides that the fermentation residues dewatered by means of the vertical press are thermally utilized, which includes all types of thermal utilization such as, for example, pyrolysis, but in particular preferably combustion. The resulting heat can then be used in whole or in part to operate the fermentation plant. Thus a cycle process is made possible here, with which the biogenic material can be utilized with particularly high efficiency. Particularly in the field of biogas plants, which increasingly rely on methanization, i.e. the direct utilization of the biogases as gas and not, for example, - via the operation of gas engines - as electrical energy, there is a heat deficit for the operation of the biogas plant due to the omission of otherwise available engine waste heat, so that the arrangement proposed here makes the operating form of methanization possible in real terms only(enabling technology).

Particularly preferred is the use of an device or method for dewatering a mass of material according to the first aspect of the invention within a method or system for utilizing a biogenic material according to the second aspect of the invention, i.e., the use of the vertical press with a press space open in the direction of the feed and discharge side for pressing the fermentation residues.

### BRIEF DESCRIPTION OF THE DRAWINGS

In the following, embodiments are described in more detail with reference to drawings. Thereby show:

FIG. 1 in schematic side view an embodiment of a dewatering device with a vertical press;

FIG. 2 in side view, partially in elevation, a tool of the vertical press of FIG. 1;

FIG. 3a a schematic front view of the device from FIG. 1;

FIG. 4a a schematic side view of a plant with a dewatering device according to FIGS. 1-3;

FIG. 5 schematically an embodiment of a system and method for the utilization of a biogenic material

FIGS. 6, 7 in schematic representation a first and a second alternative embodiment of a dewatering device with a vertical press.

### DETAILED DESCRIPTION OF EMBODIMENTS

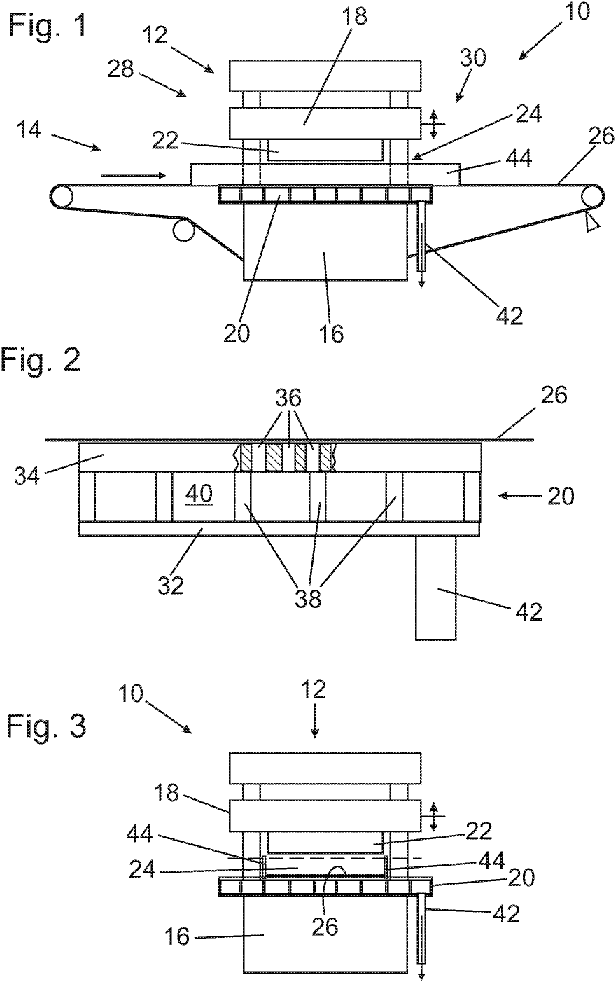

FIG. 1 shows a side view of an embodiment of a dewatering device 10 with a vertical press 12 and a conveyor 14.

The vertical press 12 comprises a press table 16, opposite which a press ram 18 is vertically guided and movable by a press drive (not shown). The press drive and the conveyor 14 are controlled by a process control system (not shown).

A tool 20 rests on the press table 16. The press ram 18 is provided with a punch 22. A press space 24 is formed between the punch 22 and the tool 20, which has a different height relative to the tool 20 depending on the stroke position of the press ram 18 and punch 22. The tool 20 is larger in length and width than the punch 22, thus protruding in all lateral directions beyond the press space 24.

The conveyor device 14 comprises an endless conveyor belt 26 which runs in a conveying direction indicated by an arrow in FIG. 1 from a feed side 28 of the press space 24 on the left in FIG. 1 to a discharge side 30 of the press space 24 arranged on the right in FIG. 1 and is returned outside the press space 24. The conveyor belt 26 is designed to be permeable to liquid and is guided over guide rollers, a drive roller and a tension roller.

FIG. 2 shows the structure of the tool 20, which comprises a closed base plate 32 and a perforated plate 34 arranged parallel to and at a distance from it and having openings, in this case round bores 36. Between the base plate 32 and the perforated plate 34, exemplary plate-shaped support elements 38 are arranged, between which a tool space 40 remains free. The tool space 40 is closed to the sides (and shown open in the drawings for illustrative reasons only). A downpipe 42 is attached to the base plate 32 and connected to the tool space 40.

FIG. 3 shows the dewatering device 10 in front view, i.e. from the feed side 28. As shown there, the press space 24 is confined laterally of the conveyor belt 26 by side walls 44. The plunger 22 is in a lower position between the side walls 44. However, towards the feed side 28 and discharge side 30 the press space 24 is open over its entire height and width. The side walls 44 extend between the feed side and discharge side and, in the embodiment shown, are longer than the press space 24 so that they project beyond the press space 24 on both sides.

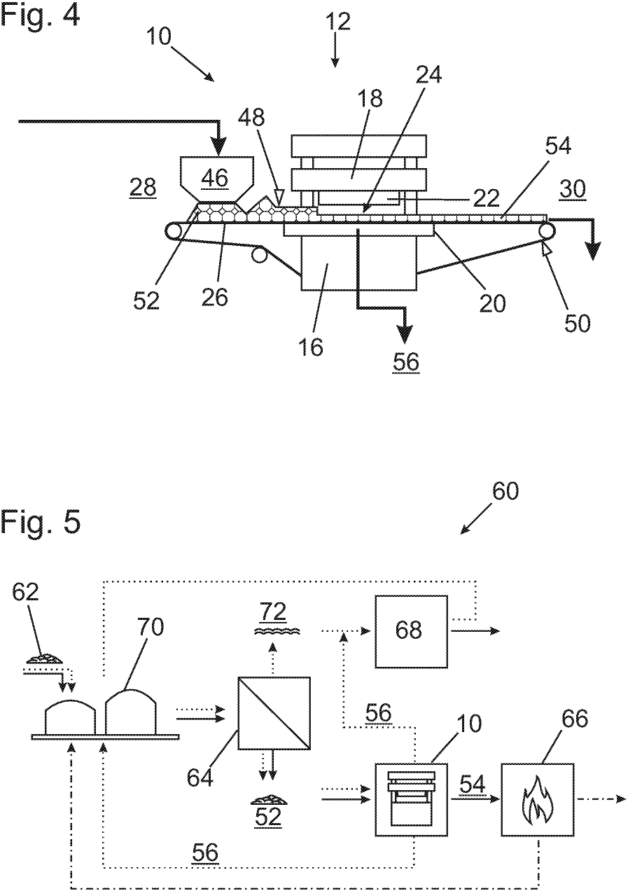

FIG. 4 shows the dewatering device 10 as part of a system for dewatering a mass of material with a liquid component, in this case in particular fermentation residues 52 from a fermentation plant or biogas plant 70 (see FIG. 5).

A bulk material feeder 46 serves to receive and meter the fermentation residue mass 52 onto the conveyor belt 26. By means of the conveyor belt 26, the fermentation residue mass 52 is conveyed from the feed side 28 into the press space 24 of the vertical press 12. A scraper 48 serves to limit a filling height within the press space 24.

After the fermentation residue mass 52 is introduced into the press space 24, it is pressed between the punch 22 and the tool 20 and thus dewatered. The liquid contained in the mass drains off through the liquid-permeable conveyor belt 26 and the openings 36 on the upper side of the tool 20 (see FIG. 2). During pressing, the side walls 44 (see FIGS. 1, 3; not shown in FIG. 4 for clarity) act as boundaries preventing lateral escape of the fermentation residue mass 52. In the direction of the feed and discharge sides 28, 30, the press space 24 is open, but escape of the fermentation residue mass is limited by the portions thereof resting in front of and behind it on the conveyor belt 26.

In the embodiment shown, the punch 22 and the sidewalls 44 each have planar, closed surfaces so that the pressed out liquid 56 drains only through the perforated plate 34 and from there passes through the tool space 40 into the downpipe 42. In alternative embodiments, grooves or through openings may be formed in the surfaces of the punch 22 and/or the side walls 44 to assist in draining the liquid.

Due to the lateral projection of the tool 22 over the press space 24, liquid components that may occur laterally from the press space 24 are also collected and pass through the tool space 40 into the downpipe 42.

After the pressing process, the pressed, dewatered fermentation residue mass 54 is conveyed by the conveyor belt 26 to the discharge side 30, where a scraper 50 assists in separation from the conveyor belt 26.

In this case, the operation of the plant and the dewatering device 10 is clocked by a control system (not shown), i.e. the advance through the conveyor belt 26 is not continuous. Instead, in each cycle step, the unpressed fermentation residue mass 52 is fed in at a predetermined maximum filling level, followed by a pressing operation by lowering the ram 22 and, after the ram 22 has been raised, the pressed fermentation residue mass 54 is conveyed on to the discharge side 30. The stroke of the press ram 18 and the ram 22 follows a movement profile predetermined by the control system with set parameters for the course of the press speed and the applied pressure.

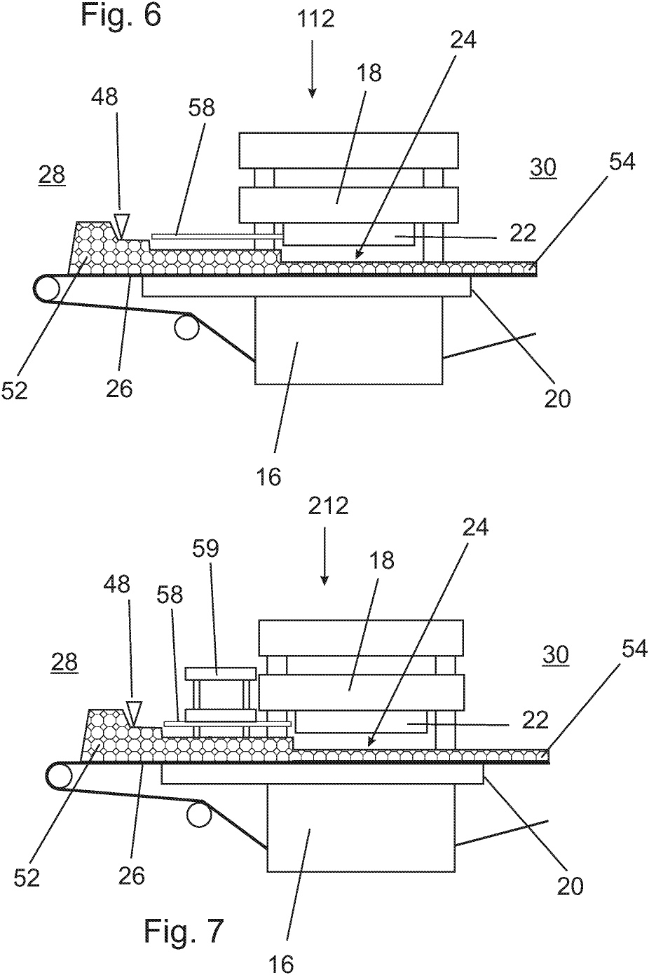

FIGS. 6, 7 illustrate further embodiments of vertical presses 112, 212 for use in a dewatering device 10. Here, the vertical presses 112, 212 largely correspond to the vertical press 12 according to the embodiment described above. Identical elements are indicated by identical reference signs. In the following, only the differences will be discussed in more detail.

In the vertical press 112 in FIG. 6, a pre-compaction plate 58 is arranged on the press plunger 22, which projects in the direction of the feed side 28. The pre-compaction plate 58 has the same length and width as the press ram 22, but its lower surface facing the fermentation residue mass 52 is displaced relative to the lower surface of the press ram 22, namely arranged higher by a distance.

This arrangement serves to pre-compact the fermentation residue mass 52 and thus to further reduce the filling height of the fermentation residue mass 52 on the conveyor belt 26. When the press plunger 22 moves downwards during the pressing process and the fermentation residue mass 52 is pressed in the press space, the pre-compacting plate 58 compresses the fermentation residue mass 52, which has already been applied to the conveyor belt 22 for feeding, by a certain amount. In this way, the necessary stroke of the press plunger 22 can be limited and particularly well utilized. Due to the vertical distance between the lower surface of the press plunger 22 and the lower surface of the pre-com-paction plate 58, the pressing pressure during pre-compaction is lower than during the subsequent pressing process.

In the vertical press 212 in FIG. 7, a pre-compaction plate 58 is also provided, but in this example it is driven by a pre-compaction press 59 independently of the press plunger 22. Thus, the entire press power is available for the pressing process.

FIG. 5 shows an embodiment of a system 60 for the utilization of biomass 62, for example agricultural waste such as liquid manure with portions of energy crops. The system 60 comprises, in addition to the dewatering device 10, for example according to the embodiment described above, a fermentation plant (biogas plant) 70, a solid-liquid separation 64, a combustion chamber 66 and a fertilizer processing 68.

In the schematic representation in FIG. 5, the transfer of solids is shown with solid lines, of liquids with dotted lines, and of heat with dash-dotted lines.

In the biogas plant 70, methane-containing biogas is produced from the biomass 62 with the addition of heat and, if necessary, additional liquid. Liquid and solid fermentation residues remain from the biomass 62, from which a liquid phase 72 is separated in the solid-liquid separation 64. The liquid phase 72 can be processed in the fertilizer processing unit 68, for example, to produce mineral fertilizer.

In addition to the liquid phase 72, a solid phase 52 is separated in the solid-liquid separation 64, namely the fermentation residues 52. These further have a high liquid content or low dry substance content of, for example, 15 to 35%. By means of a vertical press, in particular preferably as described above with regard to FIG. 4, the dry substance content is substantially increased in a very short time to, for example, 60 to 70% by pressing. Depending on the embodiment, the press liquid 56 obtained and discharged during the pressing process can either be partially or completely returned to the fermentation plant 70, fed to the fertilizer processing 68 or otherwise utilized.

Pressing enables thermal utilization of the pressed fermentation residues 54. These are thermally utilized in the combustion chamber 66, in particular by combustion. Depending on the embodiment, the resulting heat can be returned in whole or in part to the fermentation plant 70 to operate the biogas process and/or used in whole or in part elsewhere, for example as district heating.

The system 60 according to FIG. 5 and the method carried out therein exhibit particularly high efficiency in the utilization of the biomass 62, in particular by the pressing process in the dewatering device 10. Although this does not achieve complete drying of the fermentation residue mass 52, it does achieve such a targeted increase in the dry substance content that the subsequent thermal utilization in the combustion chamber 66 is made possible. By the feedback, namely on the one hand use of the heat of the thermal utilization and on the other hand return of the press liquid 56 to the biogas plant 70, an advantageous cycle process is implemented.

1. Device for dewatering a mass of material, comprising
a vertical press with a press space formed between a tool provided with openings and a press plunger movable relative to the tool,
conveying means for conveying the mass of material from a feed side into the press space and for conveying the pressed mass of material in the direction of a discharge side out of the press space opposite the feed side,
wherein the press space is laterally confined by opposite side walls extending between the feed side and the discharge side, and wherein the press space is not laterally confined in the direction of the feed side and the discharge side.

2. Device of claim 1, wherein
the conveying means comprise a liquid-permeable conveyor belt which extends from the feed side to the discharge side and rests on the tool in the press space.

3. Device of claim 2, wherein
the conveyor belt is closed by a return outside the press space (24).

4. Device according to claim 1, wherein
the side walls have a surface with openings and/or grooves in the direction of the press space.

5. Device according to claim 1, wherein
the press plunger has a surface with openings and/or grooves in the direction of the press space.

6. Device according to claim 1, wherein
the tool comprises a perforated plate with the openings, a base plate and a support frame arranged therebetween, so that a flow space for discharging liquid through the openings and the interior of the support frame is formed.

7. Device according to claim 1, wherein
collection means and at least one conduit are provided for collecting and directing liquid forced through the openings.

8. Device according to claim 1, wherein
a scraper is arranged on the feed side to limit a filling level in the press space.

9. Device according to claim 1, wherein
an arrangement for pre-compacting the mass of material is arranged on the feed side.

10. Method of dewatering a mass of material, in which
the mass of material is conveyed from a feed side into the press space of a vertical press
and the mass of material in the press space is pressed by movement of a press plunger relative to a tool provided with openings, so that at least a part of a contained liquid drains off through the openings,
and the pressed mass of material is conveyed to a discharge side opposite the feed side out of the press space,
wherein the press space is laterally confined by opposite side walls extending between the feed side and the discharge side, and wherein the press space is not laterally confined in the direction of the feed side and the discharge side.

11. Method of utilization a biogenic material in which
the biogenic material is converted microbially and/or enzymatically into gas and fermentation residues in a fermentation plant,
and the fermentation residues are dewatered by a vertical press to such an extent that thermal utilization of the dewatered fermentation residues is made possible.

12. Method according to claim 11, wherein
the fermentation residues are dewatered by the vertical press to a dry substance value of 50 to 90%.

13. Method according to claim 11, wherein
the press water pressed out by the vertical press is fed to the fermentation plant and/or to a treatment plant.

14. Method of claim 11, wherein
the dewatered fermentation residues are thermally utilized,
and the resulting heat is used to operate the fermentation plant.

15. Method of claim 11,wherein
the fermentation residues are dewatered by a device according to claim 1.

16. System for recycling a biogenic material (62), with
a fermentation plant for microbial and/or enzymatic conversion of the biogenic material into gas and fermentation residues,
and a vertical press for dewatering the fermentation residues in such a way that thermal utilization is possible.

17. Method according to claim 11, wherein
the fermentation residues are dewatered by the vertical press to a dry substance value of 55 to 80.

18. Method according to claim 11, wherein
the fermentation residues are dewatered by the vertical press to a dry substance value of 60 to 75%.
